SEQUENCE LISTING (4 sequences)

SEQ ID NO: 1 - DNA, 17 bp, Artificial Sequence
  Description of Artificial Sequence Synthetic
tccccatcgccatcccc

SEQ ID NO: 2 - DNA, 18 bp, Artificial Sequence
  Description of Artificial Sequence Synthetic
caccatggcctcggctgg

SEQ ID NO: 3 - DNA, 30 bp, Artificial Sequence
  Description of Artificial Sequence Synthetic
tgacggggtcacccacactgtgcccatcta

SEQ ID NO: 4 - DNA, 30 bp, Artificial Sequence
  Description of Artificial Sequence Synthetic
ctagaagcatttgcggtggacgatggaggg

(19) United States
(12) Patent Application Publication
(10) Pub. No.: US 2002/0165188 A1
(43) Pub. Date: Nov. 7, 2002

(54) Methods for inhibition of tumorigenic properties of melanoma cells

(76) Inventors: Meenhard Herlyn, Wynnewood, PA (US); Kapaettu Satyamoorthy, Swarthmore, PA (US)

Correspondence Address: Licata & Tyrrell P.C., 66 E. Main Street, Marlton, NJ, 08053, US

(21) Appl. No.: 10/059,828

(22) Filed: Jan. 30, 2002

Related U.S. Application Data
(63) Continuation-in-part of application No. 09/686,257, filed on Oct. 11, 2000, now abandoned.
(60) Provisional application No. 60/159,353, filed on Oct. 14, 1999.

(51) Int. Cl. A61K 48/00; A61K 39/395

(52) U.S. Cl. 514/44; 424/145.1

(57) ABSTRACT

The present invention provides a method for preventing proliferation of melanoma cells by contacting melanoma cells with an agent which is capable of modulating the expression of E-cadherin in the melanoma cells thereby restoring keratinocyte control over melanoma cell proliferation.

This application is a continuation-in-part of U.S. Ser. No. 09/686,257 filed Oct. 11, 2000 which claims the benefit of priority of U.S. Ser. No. 60/159,353 filed Oct. 14, 1999.

This research was supported in part by U.S. government funds from the National Cancer Institute grant numbers CA76674, CA25874. The United States government may have certain rights in the invention.

### BACKGROUND OF THE INVENTION

Melanoma is a relatively common cancer. The incidence of cutaneous melanoma has risen rapidly in the last several decades (Parker et al., 1997, C A Cancer J. Clin 47:5-27; Ries et al., 2000, Cancer, 88:2398-424). Melanoma is notorious for its propensity to metastasize and its poor response to current therapeutic regimens. The transition from benign lesions to invasive, metastatic cancer occurs through a complex process involving changes in expression and function of oncogenes or tumor suppressor genes (Meier et al., 1998, Am. J. Pathol. 156:193-200).

In the human epidermis, melanocytes residing at the basement membrane are interspersed among basal keratinocytes. E-cadherin is physiologically expressed on the cell surface of keratinocytes and melanocytes, and is the major adhesion molecule (Hsu et al., 1996, J. Investig. Dermatol. Symp. Proc. 1:188-94; Tang et al., 1994, J. Cell Sci. 107:983-92). A progressive loss of E-cadherin expression occurs during melanoma development (Danen et al., 1996, Melanoma Res., 6:127-31; Hsu et al., 1996, J. Investig. Dermatolo Symp. Proc. 1:188-94; Scott & Cassidy, J. Invest Dermatol., 1998, 111:243-50; Silye et al., 1998, J. Pathol. 186:350-55). Under natural conditions, melanocytes express E-cadherin on their surface but melanoma cells do not (Hsu et al., 1994, J. Invest. Dermatol. Symp. Proc. 1:188-194). Additionally, melanoma cells express N-cadherin, while melanocytes do not. Melanoma cells express greater amounts of Mel-CAM and αᵥβ₃ than do melanocytes. However, both cell types express α-catenin, β-catenin and plakoglobin (Ozawa et al., J. Cell Biol., 1992, 116:989-996; Knudsen et al., 1995, J. Cell Biol. 130:67-77). Growth, proliferation, dendricity and cell-surface molecule composition of melanocytes are normally under the control of basal layer-type keratinocytes (Herlyn et al., 1987, Cancer Res. 47:3057-3061; Valyi-Nagy et al., 1993, Lab. Invest 69:152-159,; Shih et al., Am. J. Of Pathol., 1994, 145:837-845). Melanoma cells are refractory to the regulatory controls normally exerted by keratinocytes and therefore proliferate in an uncontrolled manner. Isolated and cultured melanocytes lose their normal phenotype, but regain it upon co-culture with basal layer type keratinocytes. The homeostatic effects of basal layer-type keratinocytes exert these effects upon melanocytes.

Increased proliferation of human melanocytes in vitro by HGF has previously been reported (Halaban et al., 1992, Oncogene 7:2195-206; Imokawa et al., 1998, Biochem J. 330:1235-9; Matsumoto et al., 1991, Biochem Biophys Res Commun 176:45-51), however, the mitogenic activity became obvious only when HGF acted together with other growth factors such as bFGF (Halaban et al., 1992, Exs, 65:329-39). It has been previously shown that c-Met is co-localized with β-catenin and E-cadherin at regions of cell to cell contact in human colon and breast cancer cell lines (Hiscox and Jiang, 1999, Biochem Biophys Res Commun.,261:406-11 ; Kamei et al., 1999, Oncogene 18:6776-84). Previous studies have shown a progressive loss of E-cadherin expression during melanoma development (Hsu et al.,1996, J. Investigat. Dermatol. Symp. Proc.; Johnson, 1999, Cancer Metastasis Rev.18:345-57). Physiologically, HGF is secreted by cells of mesenchymal origin and acts as mitogen, motogen and morphogen for many epithelial cells (Gherardi et al., 1989 Proc. Natl. Acad. Sci. USA, 86:5844-8; Nakamura et al., 1989 Nature 342:440-3; Stoker et al., 1987, Nature, 327:239-42) and is therefore considered a paracrine factor. Until the present invention it was previously unknown how c-Met is activated or the source of its ligand, HGF.

One important aspect of HGF is its mitogenic activity (Vande Woude et al., 1997, Ciba Found Symp. 212:119-30). However, a significant increase in melanocytic cell proliferation was not detected after either pulse or prolonged HGF stimulation, suggesting that for melanocytic cells, HGF is not a potent mitogen. (Halaban et al., 1993, Exs, 65:329-339; Rusciano et al., 1998, Tumour Biol. 19:335-45; Halaban et al., 1992, Oncogene,7:2195-206; Tamatani et al., 1999, Carcinogenesis, 20:957-62). IF-1 (Radix et al., 1987, INT. J. Cancer, 40:687-90) and bFGF (Halaban et al., 1992, Oncogene, &:2195-206; Meier et al., 2000, Am. J. Pathol., 156:193-200) are the most important mitogens for melanocytic cells.

Desmosomes contain specialized cadherin adhesion molecules (Desmogleins and Desmocollins) associating with various cytoplasmic proteins such as Desmoplakins and Plakoglobin. Desmosomes provide the cells with binding domains for intermediate filaments of the cytokeratin network and are required for tissue organization (Bornslaeger et al., 1996, J. Cell Biol. 134:985-1001; Kouklis et al., 1994, J. Cell Biol. 127:1049-60; Kowalczyk et al., 1997, Ins. Rev. Cytol. 185:237-302; Smith & Fuchs, 1998, J. Cell Biol. 141:1229-41; Stappenbeck & Green, 1992, J. Cell Biol. 116:1197-209). Plakoglobin (y-catenin) is also part of the cadherin-catenin complex in adherens junctions (Aberle et al., 1994, J. Cell Sci. 107:3655-63; Butz & Kemler, 1994, FEBS Lett. 355:195-200; Lewis et al.,1997, J. Cell Biol. 136:919-34) and may mediate crosstalk between adherens junctions and desmosomes (Lewis et al., 1997, J. Cell Biol. 136:919-34). Reduction or loss of desmosomes may contribute to the invasive and metastatic behavior of various tumors, for example transitional cell carcinoma of the bladder (Conn et al., 1990, Br. J. Urol. 65:176-80) and squamous cell carcinomas (Depondt et al.,1999, Eur. J. Oral Sci. 107:183-193; Krunic et al., 1996, Acta Derm. Venereol. 76:394-8,; Shinohara et al., 1998,c. Reduction in desmosome formation was correlated with invasion and with reduction in E-cadherin staining (Shinohara et al., 1998 J. Pathol. 184, 369-81). Desmosomes are also believed to play an important role in maintaining human skin homeostasis. Until the present invention it was unclear which, if any, of these proteins was able to induce or inhibit one or more tumorigenic properties in a melanocyte.

### SUMMARY OF THE INVENTION

The present invention provides a method for preventing proliferation of melanoma cells comprising contacting melanoma cells with an agent which modulates expression of E-cadherin in melanoma cells thereby restoring keratinocyte control over melanoma cell proliferation.

The present invention further provides a method of preventing proliferation of melanoma cells comprising contacting melanoma cells with an agent which modulates the HGF autocrine loop in melanoma cells to prevent down regulation of E-cadherin and Desmoglein 1, wherein said method prevents uncontrolled proliferation of said melanoma cells and dissemination of tumor masses.

The present invention further relates to a method of treating melanoma comprising administering an effective amount of an agent to melanoma cells, which agent prevents constitutive activation of c-Met, MAPK, AKT or PI3K in said melanoma cells.

The present invention further relates to a method of preventing proliferation of melanoma cells comprising contacting melanoma cells with an agent which modulates expression of cadherin in melanoma cells wherein genes which control or transmit physiologically relevant signals from keratinocytes to melanocytes are upregulated or down regulated so that control over melanoma cell proliferation is restored.

### DETAILED DESCRIPTION OF THE INVENTION

The present invention is based on the surprising discovery that enhanced expression of E-cadherin in melanoma cells restores keratinocyte control over melanoma cell proliferation, growth and dendricity, even over highly aggressive and metastatic melanoma cells. Enhanced expression of E-cadherin may be achieved through an agent which directly modulates the presence of E-cadherin in a melanoma cell or through modulation of another compound which prevents the down-regulation of E-cadherin in a melanoma cell. Thus, expression of E-cadherin in melanoma cells mediates resumed control of melanoma cell proliferation by keratinocytes. Enhanced expression in melanocytes of E-cadherin has been found to induce diminished expression of the melanoma associated cell surface proteins Mel-CAM (MUC18) and αᵥβ₃ when the melanoma cells are co-cultured with keratinocytes. These cell surface proteins are expressed on melanoma cells but are expressed at a much lower level, or not at all, on non-tumorigenic melanocytes. Thus, when the melanoma cell is cocultured with keratinocytes the invasiveness of the cell is reduced. Restoration of E-cadherin expression in melanoma cells results in restored keratinocyte-mediated growth control and downregulated expression of invasion-related adhesion receptors (Hsu et al., 2000, Am. J. Pathol. 153:1435-42). It is believed that expression of E-cadherin renders melanoma cells susceptible to physiological control signals which keratinocytes normally exert upon melanocytes. Such controls may be exerted upon melanocytes. Such controls may be exerted upon melanocytes either by a biological activity catalyzed by E-cadherin protein of the melanocytes or by a protein to which E-cadherin is able to indirectly transmit a signal, e.g. by way of intracellular signaling proteins such as α-catenin, β-catenin , plakoglobin, desmoplakins, desmogleins, desmocollins. Alternatively, the interaction between E-cadherin and a keratinocyte enables the keratinocyte to affect the biological activity of another melanocyte protein which generates or transmits a control signal to melanoma-associated melanocyte proteins.

There is a distinction between the use of the terms melanocyte and melanoma cell. As used herein, a melanocyte is a normal non-tumorigenic cell. A melanoma cell is a non-normal tumorigenic melanocyte. A tumorigenic melanocyte is one which exhibits uncontrolled growth and invasive characteristics which are not evident in melanocytes.

Several proteins are believed to be involved in transmission of physiologically relevant signals from keratinocytes to melanocytes. Proteins which are believed to be important in this regard are E-cadherin, α-catenin, β-catenin, plakoglobin, p120 (ctn), an axin, a glycogen synthesis kinase, APC, PKB/Akt, an Lef/TCF protein, a POU domain containing protein, an Ets protein, an E-box binding protein, a protein of the myc family, a protein having one or more leucine zipper motifs, a homeobox protein, a protein having one or more homeobox motifs, Shc, a helix-loop-helix protein, a basic helix-loop-helix leucine zipper containing protein, a protein of the disheveled and frizzled family of proteins, a protein which modulates apoptosis, Bcl-2, Bax and Bad.

Enhanced expression of E-cadherin in melanoma cells facilitates the keratinocyte induced inhibition of tumorigenic properties of the melanoma cells. Expression of E-cadherin in a melanoma cell results in reduced expression of Mel-CAM and αᵥβ₃ in the cell. In addition, enhanced expression of E-cadherin in melanoma cells is also believed to result in modulation of specific genes which are believed to be involved in the control or transmission of physiologically relevant signals from keratinocytes to melanocytes. The specific genes identified which are believed to be important in this regard are and which are upregulated or down regulated with cadherin expression are provided in Tables 1 and 2 of Example 11.

Besides E-cadherin-mediated adherens junctions, desmosomes are also believed to play an important role in maintaining human skin homeostasis. Desmosomes appear to have tumor-suppressor properties. In fact, over expressing desmosomal cadherins in squamous cell carcinoma cells inhibited invasiveness (De Bruin et al.,1999, Cell Adhes Commun, 7:13-28; Tada et al, 2000, J. Cutan Pathol. 27, 24-29).

Hepatocyte growth factor (HGF) is a multi-functional cytokine which acts as mitogen, motogen and morphogen for many epithelial cells through its tyrosine kinase receptor c-met which is present in ipithelial cells and malanocytes. HGF is physiologically secreted by cells of mesenchymal origin and acts on neighboring epithelial cells through a paracrine loop. However, coexpression of HGF and c-Met has been identified in a variety of transformed cells and tumors both in vitro and in vivo and shown to be involved in tumor development and invasion.

HGF/c-Met signaling has been implicated in the potential development of melanoma (Hendrix et al., 1998, Am J. Pathol. 152:855-63; Natali et al., 1993 Br. J. Cancer 68:746-750; Rusciano et al, 1998, Tumour Biol. 19:335-45). HGF stimulates proliferation and motility of human melanocytes (Halaban et al., 1992 Oncogene 7:2195-206; Imokawa et al., 1998 Biochem J. 330:1235-9; Kos et al., 1999 Pigment Cell Research 12:13-21). In transgenic mice that ubiquitously expressed HGF, ectopic localization of melanocytes and hyperpigmentation in skin were observed (Takayama et al., 1996 Proc Natl Acad Sci USA 94:701-6) and melanoma arose spontaneously (Kos et al., 1999 Pigment Cell Research 12:13-21; Otsuka et al., 1998, Cancer Research 58:5157-67; Takayama et al., 1996, Proc. Natl Acad Sci USA 93:5866-71). In these mice, ultraviolet radiation-induced carcinogenesis was accelerated (Noonan et al., 2000, Cancer Res 60:3738-43). It is suggested that c-Met autocrine activation induced development of malignant melanoma and acquisition of the metastatic phenotype (Otsuka et al., 1998, Cancer Research 58:5157-67). However it was not until the present invention that the autocrine loop was shown to actually exist in human melanoma. Further, until the present invention the mechanism by which HGF induces human melanoma were unclear.

The present invention provides a method of treating human melanoma by preventing proliferation of melanoma cells comprising contacting melanoma cells with an agent which modulates expression of E-cadherin and restores keratinocyte control over melanoma cells.

In one aspect, the present invention provides a method of preventing proliferation of melanoma cells comprising contacting melanoma cells with an agent which modulates the HGF autocrine loop in melanoma cells to prevent down regulation of E-cadherin and Desmoglein 1 to prevent uncontrolled proliferation of melanoma cells and dissemination of the melanoma cells into tumor masses. The HGF autocrine loop has been found to disrupt adhesion between melanocytes and keratinocytes by downregulating E-cadherin and Desmoglein 1, resulting in decoupling melanocytic cells from the control by keratinocytes, which is believed to allow uncontrolled proliferation of cancer cells and dissemination of the tumor mass. It has been found that Desmoglein 1 is downregulated in melanoma cell lines, and Desmoglein 1 expression levels correlate with those of E-cadherin. During melanoma development, there is a progressive loss of E-cadherin expression. It is believed that cell to cell adhesions must be disrupted before malignant cells can migrate and metastasize to remote locations; and that besides E-cadherin-mediated adherens junctions, other types of adhesive junctions have to be attenuated as well. Since desmosomes are important in maintaining human skin homeostasis, they are a major target of cell to cell detachment during transformation. Western blots have shown that both keratinocytes and primary melanocytes expressed high levels of Desmoglein 1, while the majority of melanoma cells i.e., 18 out of 20, expressed very little Desmoglein 1. Two exceptions, however, are melanoma cell lines, WM35 and WM164, which express Desmoglein 1 at levels comparable to those in keratinocytes or melanocytes. These two cell lines, WM35 and WM164, are exceptional in that unlike other melanoma cells, they are E-cadherin positive, suggesting that there is a correlation between the levels of E-cadherin and those of Desmoglein 1 in melanoma cells. Desmoglein 2 and Desmoglein 3 were not detectable by Western blots in either normal melanocytes or melanoma cells of various stages. Since Plakoglobin (y-catenin) is shared by both adherens junctions and desmosomes and murine melanoma cell lines suffer a loss of Plakoglobin, Plakoglobin expression was tested in melanoma cells. Although reduced expression and absence were seen in some cell lines there was no clear trend. Immunofluorescence staining showed that in Desmoglein-positive cells (FOM73 and WM35), Desmoglein 1 was concentrated at the cell-cell contact areas whereas in Desmoglein-low cells such as 1205Lu, no distinct staining was observed.

Desmoglein 1 functions as a co-receptor for E-cadherin in mediating cell to cell adhesion between melanocytes and keratinocytes. It was demonstrated that desmosomes function together with adherens junctions to mediate cell to cell adhesion between melanocytes and keratinocytes, dominant negative E-cadherin (E-cadΔEC) and Desmoglein 3 (Dsg3ΔEC) was expressed in melanocytes. Dsg3ΔEC targets Desmoglein 3, and is also able to disrupt Desmoglein 1 as well. After confirming Desmoglein 1 and Desmoglein 3 expression in melanocytes, cell adhesion assays were used to evaluate the role of Desmoglein 1 and Desmoglein 3 in melanocyte-keratinocyte interactions. E-cadΔEC significantly disrupted adhesion between the two cell types exhibiting a 42% reduction, whereas Dsg3ΔEC alone did not make a significant difference. When both Dsg3ΔEC and E-cadΔEC were introduced to melanocytes, a strong disruption, about a 79% reduction, was obtained. This finding indicates that disruption of Desmoglein alone is apparently not enough to break cell to cell adhesion, however it can further weaken the interaction if E-cadherin-mediated adhesion is also lost.

Gene deletion or promoter methylation was not detected in the melanoma lines evaluated. This lack of deletion or promoter methylation is in contrast to what has been shown in other cancer types but is consistent with observations in melanoma. Therefore, abnormal growth factor expression was investigated to determine whether it affected the downregulation of E-cadherin and/or Desmoglein.

Melanoma cells, but not normal melanocytes, express HGF, which causes constitutive activation of c-Met, MAPK and P13K in melanoma cells. During screening of growth factors, it was found that the majority of melanoma cells express HGF. In normal skin, HGF is secreted by cells of mesenchymal origin such as fibroblasts, but not melanocytes. Therefore, it was surprising to find that the HGF autocrine loop exists in melanoma cells. The two lines (WM35 and WM164) with high E-cadherin and Desmoglein 1 expression showed little HGF expression. Further testing of the expression and status of the HGF receptor, c-Met, showed that all cells, including keratinocytes, melanocytes and melanoma cells, expressed c-Met and there was no strong correlation between HGF expression levels and c-Met abundance. When melanocytes and melanoma cells were treated with additional HGF, a dramatic increase in tyrosine-phosphorylated c-Met was found. This increase in tyrosine-phosphorylated c-Met suggests that c-Met is functional in these cells. The levels of c-Met did not change after HGF treatment. Constitutively phosphorylated c-Met could be detected even in the absence of exogenous HGF as found in cell lines WM278 and 1205Lu. The constitutive activation is caused by endogenous HGF, because a HGF blocking antibody reduced c-Met phosphorylation. Since WM164 and WM35 expressed little HGF, the endogenous levels of c-Met activation were low. Melanocytes contained no constitutively active c-Met as shown by cell line FOM73. Constitutive activation of MAPK and AKT was observed in melanoma both in vitro and in vivo. To demonstrate that autocrine HGF is, at least in part, responsible for constitutive activation, a HGF blocking antibody was used to treat 1205Lu melanoma cells. After HGF neutralization, a significant decrease in phosphorylation of both MAPK and AKT was observed.

In another aspect, the present invention provides a method of preventing proliferation of melanoma cells comprising contacting the melanoma cells with an agent which modulates the HGF autocrine loop so as to down regulate E-cadherin and Desmoglein 1. This type of down regulation prevents uncontrolled proliferation of melanoma cells. Effective agents are antisense oligonucleotides, HGF blocking antibody or other blocking antibodies.

In another aspect, the present invention provides a method of treating melanoma by administering an effective amount of an agent to melanocytes or melanoma cells which prevents or interrupts prolonged HGF stimulation thereby preventing down regulation of E-cadherin and Desmoglein 1. Sustained HGF stimulation has been found to cause down-regulation of both E-cadherin and Desmoglein 1 in melanocytic cells through P13K and MAPK pathways. No significant growth stimulation in either melanocytes or melanoma cells after HGF treatment alone was found. Surprisingly, a dramatic decrease in the expression levels of Desmoglein 1 and E-cadherin were detected in melanocytes, WM164 and WM35 melanoma cells, exposed to continuous HGF stimulation for three days. An adenoviral vector Ad.CMV.rhHGF (Phaneuf et al., 2000, Mol. Med 6:96-103) was then used at 20 pfu/cell (corresponding to 30 ng/ml at 72 hours) to create an autocrine loop in these cells and to ensure sustained stimulation. Under the same conditions, expression of Connexin 43 did not change. Noticeably, stimulation of melanocytes with one pulse of recombinant protein at 50 ng/ml at five and twelve hour intervals did not alter E-cadherin or Desmoglein expression, suggesting that continuous stimulation is necessary for the observed changes. HGF stimulation can initiate multiple signal transduction pathways, including the MAPK and P13K cascades. Whether the downregulation of adhesion molecules depends on these transduction pathways was unclear. When HGF neutralizing antibodies (10 μg/ml) were added to Ad.CMV.rhHGF transduced melanocytes, downregulation of E-cadherin and Desmoglein 1 was abolished. The same effect was obtained in wortmannin (30 μM) treated cells. PD98059 (10 μM and 50 μM) was less effective in blocking the downregulation, indicating that although both P13K and MAPK are involved, the P13K pathway is the more critical pathway.

Desmoglein 1, E-cadherin, c-Met and Plakoglobin can be co-immunoprecipitated from melanocytic cells. The initial immunofluorescence assays showed that c-Met, E-cadherin and Desmoglein 1 are co-localized at the cell surface of melanocytes. Since all of the above molecules are membrane-bound proteins, membranous co-localization is not necessarily suggestive of direct physical interaction. To test the possibility that physical interactions underlie the crosstalk between HGF/c-Met signaling and adhesion receptor regulation, the complex formation was investigated by immunoprecipitation analyses. Desmoglein 1, Plakoglobin, c-Met and E-cadherin were detected in the complexes pulled down by either anti-Desmoglein 1 antibody or anti-Plakoglobin antibody in keratinocytes and melanocytes. WM278 expresses no E-cadherin, but the other components were also found to form complexes. The specificity of the interaction was confirmed by probing the same blots with anti-IF-R1 antibody. IF-R1 was not detected in the immunoprecipitants but IF-R1 was detectable in whole cell extract.

There is a functional importance of cell adhesion molecules in normal skin homeostasis and in melanoma development (Hsu et al.,2000, Am. J. Pathol. 156:1515-25; Johnson, 1999, Cancer Metastasis Rev. 18:345-357; Li & Herlyn, 2000, Mol. Med. Today 6:163-69).

The present invention provides evidence that cross talk between cell adhesion molecules and growth factor HGF is responsible for dysregulated skin homeostasis and tumor development. Specifically, in the present invention Desmoglein 1 functions as a co-receptor for E-cadherin and mediates cell adhesion between melanocytes and keratinocytes. Although both E-cadherin and Desmoglein 1 are highly expressed in normal melanocytes, they are absent or substantially reduced in melanoma cells. Melanoma cells, but not normal melanocytes, express HGF, which causes sustained activation of the HGF receptor c-Met and its downstream effectors MAPK and AKT/PKB. Further, prolonged HGF stimulation causes downregulation of E-cadherin and Desmoglein 1, which is MAPK- and P13K-dependent. Desmoglein 1, E-cadherin, Plakoglobin and c-Met can be co-immunoprecipitated from melanocytic cells, which is believed to be the pathway or mechanism(s) by which E-cadherin and Desmoglein 1 are coordinately downregulated by c-Met activation.

In the present invention, HGF can be provided by not only fibroblasts (paracrine), but also, more importantly, melanoma cells themselves (autocrine). The latter is believed to play a much more important role in melanoma development because fibroblasts constantly express HGF, even under normal conditions. Endogenously expressed HGF causes sustained activation of c-Met and its downstream effectors MAPK and P13K. Furthermore, when E-cadherin and Desmoglein-positive cells were exposed to prolonged HGF treatment, the expression levels of the two adhesion molecules were greatly reduced. Interestingly, pulse treatment with HGF did not cause downregulation of either E-cadherin or Desmoglein, suggesting that continuous stimulation is necessary for the observed changes. It has been shown previously that HGF could disrupt intercellular junctions in normal and tumor cells (Hiscox & Jiang, 1999, Biochem Biophys. Res. Commun. 261:406-11; Stoker et al., 1987, Nature, 327:239-42; Takeichi et al., 1994, Princess Takamatsu Symp. 24:28-37). However, in those cases, HGF decreased cell to cell contact without apparent loss of E-cadherin expression, which may represent a rapid effect of HGF stimulation occurring via post-translational modification.

Activation of both MAPK (p421p44) and P13K is required for HGF-induced motility. Using the MAPK inhibitor PD98059 and the P13K inhibitor wortmannin, both pathways are involved in the downregulation. It is believed that a downstream molecule directly responsible for the downregulation is a zinc finger transcription factors such as Snail and/or Slug. Slug has been shown to play a critical role in HGF-induced desmosome dissociation in a bladder carcinoma cell line (Savagner et al., 1997, J. Cell Biol., 137:1403-19). In melanoma cells, activation of Snail expression plays an important role in downregulation of E-cadherin (Poser et al., 2001, J. Biol. Chem. 25:25.) Epithelial cells that ectopically express Snail adopt a fibroblastoid phenotype and acquire tumorigenic and invasive properties. Endogenous Snail protein is present in invasive mouse and human carcinoma cell lines and tumors in which E-cadherin expression has been lost.

Besides the downregulation of E-cadherin and Desmoglein, an HGF autocrine loop in melanoma has other important implications. For example, activation of vibronectin receptor αᵥβ₃ can be induced by HGF (Trusolino et al., 1998, J. Cell Biol., 142:1145-56). Up-regulation of αᵥβ₃has been implicated in melanomas (Albelda et al., 1990, Cancer Res. 50:6757-64; Hsu et al., 1998, Am. J. Pathol. 153:1435-42; Van Belle et al., 1999, Hum. Pathol. 30:562-7). This has implications for invasion processes, particularly in the context that activation of c-Met by HGF leads to invasiveness of melanocytic cells.

The present invention also relates to pharmaceutical compositions comprising an agent which enhances expression of E-cadherin. In a preferred embodiment, enhanced expression may be achieved through use of an agent which directly modulates the presence of E-cadherin in a melanoma cell. In another preferred embodiment, enhanced expression may be achieved through administration of an agent which modulates a compound which prevents the down-regulation of E-cadherin in a melanoma cell.

The present invention also relates to a method for identifying an agent which modulates expression of E-cadherin in melanoma cells comprising contacting melanoma cells with a test agent and determining whether keratinocytes control over melanoma cell proliferation is restored.

The compositions of the present invention may comprise both an agent which directly modulates the presence of E-cadherin in a melanoma cell, and an agent which modulates a compound which prevents the down-regulation of E-cadherin in a melanoma cell in a single pharmaceutically acceptable formulation. Alternatively, the components may be formulated separately and administered in combination with one another. Various pharmaceutically acceptable formulations well known to those of skill in the art can be used in the present invention. Selection of an appropriate formulation for use in the present invention can be performed routinely by those skill in the art based upon the mode of administration and the solubility characteristics of the components of the composition.

As will be obvious to one skilled in the art upon reading of this disclosure, the methods of the present invention are particularly useful to treat melanoma cells. The invention is further illustrated by the following non-limiting examples.

### EXAMPLES

### Example 1

### Cell Culture

Human melanoma cells were isolated from clinically and histologically defined lesions (Satyamoorthy et al., Melanoma Res. 7: Supplement 2, S35-42, 1997). Cells were maintained in medium W489, a 4:1 mixture of MCDB153 and L15, supplemented with 2% heat-inactivated FBS and insulin (5 μug/ml) in a 37° C., 5% CO₂atmosphere at constant humidity. For growth in “protein-free” media, FBS and insulin were omitted from the medium. Primary human dermal fibroblasts were initiated as explant cultures from trypsin-treated and epidermis-stripped neonatal foreskin, and maintained in Dulbecco's Modified Eagle's medium (DMEM) supplemented with 10% FBS. Human melanocytes were isolated from foreskin and maintained in MCDB153 medium supplemented with FBS, endothelin-3 (ET-3) and stem cell factor (SCF). Human keratinocytes were isolated from foreskin and maintained in SFM (GIBCO BRL). Transcomplementing 293 cells, a cell line immortalized and transformed by adenovirus Ela and Elb, were obtained from the Vector Core at the Institute for Human Gene Therapy (University of Pennsylvania, Philadelphia, Pa.) and grown in DMEM with 10% FBS. All tissue culture reagents were purchased from Sigma Chemical Co. (St. Louis, Mo.) and GIBCO BRL (Gaithersburg, Md.).

### Example 2

### Antibodies and Reagents

Mouse anti-E-cadherin, -Desmoglein 1, -γ-Catenin (Plakoglobin) and -phosphotyrosine (PY20) mAbs were from Transduction Laboratories, Inc. (Lexington, Ky.). Mouse anti-β-actin antibody was purchased from Sigma (St. Louis, Mo.). Anti-IGF-R1, anti-c-Met (c-12) and anti-c-Myc (9E10) antibodies were from Santa Cruz Biotechnology Inc. (Santa Cruz, Calif.). Anti-FITC and Cy3-conjugated secondary antibodies were from Jackson Research Laboratories, Inc. (West Grove, Pa.). HGF neutralizing antibody, recombinant human HGF and HGF ELISA kit were all purchased from R&D Systems (Minneapolis, Minn.). Anti-Akt, anti-phospho-Akt (Ser473), anti-p44/42 MAPK and anti-phospho-p44/42 MAPK (Thr2021Tyr204) antibodies were purchased from Cell Signaling Technology, Inc (Beverly, Mass.). Inhibitors PD98059 and wortmannin were purchased from Sigma (St. Louis, Mo.).

### Example 3

### Adenoviral Vectors

The recombinant adenoviral vector Ad.CMV.rhHGF(Phaneuf et al., 2000 Mol. Medicine, 6:96-103), adenoviral vectors AxCANCre (Kanegae et al., 1995, Nucleic Acids Res. 23:3816-21; Kanegae et al., 1996 Gene, 181:207-12; Miyake et al., 1996 Proc. Natl. Acad. Sci. USA 93:;1320-4), AxCALNLDsg3ΔEC and AxCALNLDEcadΔEC (Hanakawa et al., 2000) were used. Plaque-purified virus propagated in 293 cells was purified by ultracentrifugation in a cesium chloride gradient. Viral titer was assayed by plaque formation in permissive 293 cells. Control adenoviral vector containing the LacZ cDNA (LacZ/Ad5) was purchased from Vector Core at the Institute for Human Gene Therapy, University of Pennsylvania, Philadelphia, Pa.

### Example 4

### Viral Infection of Melanocytes and Melanoma Cells

Optimal viral concentrations for infection were determined as the amounts of virus required to yield the desired levels of gene expression without apparent alteration in cellular phenotype or toxicity. Subconfluent melanocytes and melanoma cells were infected with adenoviruses for 2-4 hours at 37° C. in protein-free W489 medium. Viral suspension was then replaced with culture medium. Because dominant negative Desmoglein-3 vector AxCALNLDsg3ΔEC and E-cadherin vector AxCALNLEcadΔEC were made using the Cre-loxP system (Hanakawa et al., 2000), AxCANCre was co-infected with AxCALNLDsg3ΔEC or AxCALNLEcacLΔEC at a ratio of 1:1, respectively. Cells were allowed to recover for at least 24 hours before assay.

### Example 5

### Immunoblotting

For immunoblotting, cells were washed with PBS and harvested in RIPA buffer. Total protein concentrations were measured using the bicinchoninic acid (BCA) assay (Pierce Chemical Co. Rockford, Ill.). Samples were loaded at 15 μg/lane and separated on 8% SDS polyacrylamide gels and transferred to polyvinylidene difluoride (PVDF) membranes and probed with specific primary antibodies. To detect the signal, peroxidase-conjugated secondary antibody was added, followed by exposure using enhanced chemiluminescence (ECL) (Amersham, Arlington Heights, Ill.). Some of the immunoblots were quantified using NIH Image (version 1.61).

### Example 6

### Immunofluorescence

Melanocytes and melanoma cells were seeded in S-well chamber slides (LAB-TEK™, Nunc, Inc., Naperville, Ill.). Cells were washed with cold PBS, fixed in 4% paraformaldehyde at 4° C. for 5 mm, permeabilized with 0.5% Triton X-100 in PBS for 20 mm at room temperature, blocked with 5% BSA, and incubated with primary antibody (anti-Desmoglein 1gG₁, dilution 1:50), followed by biotin-conjugated goat anti-mouse IgG₁and Cy3-conjugated streptavidin. Primary and secondary antibody incubations were performed at room temperature for 1 hour with three washings in between. Cells were mounted in anti-fade medium Gel Mount (Biomeda Corp.) and examined by fluorescence microscopy.

### Example 7

### Immunoprecipitation

To investigate the status of c-Met phosphorylation, subconfluent melanoma cells were infected with LacZ or Ad.CMV.rhHGF (20 pfu/cell) and cultured for 48 hours. In neutralizing antibody-treated group, HGF neutralizing antibody (final concentration 10 ug/ml) was added to serum-free medium 24 hours before lysis. Cells were washed with cold PBS containing 1 mM Na₃VO₄, and rapidly scraped into 200 μl PBS containing 1% Triton X-100, 1% NP4O, 1 mM Na₃VO₄, 1 mM phenylmethyl sulfonyl fluoride (PMSF), and protease inhibitors (1 μg/ml leupeptin, aprotinin and pepstatin). After incubation on ice for 15 minutes, the extracts were separated by centrifugation at 13,000× g for 10 minutes. The supernatants were used for immunoprecipitation using an anti-c-Met antibody (5 μg) for 1 hour at 4° C. with shaking. Protein A-G sepharose CL-4B beads (Pharmacia Biotech, Uppsala, Sweden) were then added and incubated for 12 hours. Samples were washed four times with lysis buffer, boiled in Laemmli buffer containing (3-mercaptoethanol, and subjected to electrophoresis on an 8% SDS-polyacrylamide gel. Separated proteins were transferred onto PVDF membrane and detected by anti-phosphotyrosine primary antibody (PY2O) and peroxidase-conjugated secondary antibody. Signals were detected using ECL.

To investigate c-Met Desmoglein lE-cadherin complexes, cells cultured in their optimal media were lysed in 10 mM Tris-HCl (pH7.4), 1% NP-40, 1% Triton X-100, 2mM CaCl₂, 150 mM NaCl, 1 mM PMSF and protease inhibitors (1 μg/ml leupeptin, aprotinin and pepstatin). The extracts were immunoprecipitated using either anti-Desmoglein 1 or anti-Plakoglobin antibody in the same condition described above. Immunoprecipitants were separated on gels, transferred onto PVDF membrane and probed with anti-Desmoglein 1, -Plakoglobin, -c-Met and -E-cadherin antibodies.

### Example 8

### Detection of HGF expression by RT-PCR and ELISA

Total RNA was isolated from the cell lines using Trizol Reagent (Gibco BRL). Reverse transcription was carried using the Superscript II system (Gibco BRL). PCR primers for amplification of HGF were: forward primer 5′-TCCCCATCGCCATCCCC-3′ (SEQ ID NO:1); reverse primer 5′ CACCATGGCCTCGGCTGG-3′ (SEQ ID NO:2). The expected size of PCR product is 749 bp. Internal control primers (forward: 5′-TGACGGGGTCACCCACACTGTGCCCATCTA-3 (SEQ ID NO:3); reverse: 5′-CTAGAAGCATTTGCGGTGGACGATGGAGGG-3′ (SEQ ID NO:4)) amplified a 650 bp fragment of β-actin. PCR was performed using 1.5 U Taq DNA polymerase (Gibco BRL), 0.4 mM dNTPs (Pharmacia), 2 mM MgCl₂ in 1×PCR buffer (Gibco BRL). PCR was started with a 5 minute denaturation at 95° C., after which amplification was performed for 30 cycles of denaturation at 94° C. for 1 minute, annealing at 65° C. for 1.5 minutes, and elongation at 72° C. for 1 minute. Samples were analyzed by electrophoresis in 1.5% agarose gels containing 0.5 μg/ml ethidium bromide.

To detect secreted HGF by cells, conditioned media were collected, centrifuged to remove cellular debris, diluted to appropriate concentrations and subjected to enzyme linked immunosorbent assay (ELISA). ELISA was performed according to the conditions suggested by the manufacturer (R&D Systems, Minneapolis, MN).

### Example 9

### Cell Adhesion Assay

Melanocytes or E-cadherin-positive, Desmoglein-positive melanoma cells infected with indicated adenoviruses were pre-labeled with a fluorescent dye Dii (10 mg/ml; Molecular Probes, Eugene, OR) for 2 hours, washed with HBSS and harvested by treatment with 0.01% trypsin in HBSS containing 1 mM calcium for 30 minutes at 37° C. Under these conditions, cadherins are specifically protected from proteolytic digestion. Cells were then resuspended in assay medium (HBSS containing 1% bovine serum albumin and 1 mM calcium). About 5,000 cells were added to keratinocyte monolayer in 8-well chamber slides and allowed to adhere for 30 minutes. After removal of nonadherent cells, slides were fixed. Numbers of adherent cells per high power field in triplicate wells were counted under a fluorescence microscope.

During melanoma development, transformed cells evade keratinocyte-mediated control by downregulating cell adhesion molecules. This study investigated the regulation of cell adhesion by hepatocyte growth factor (HGF) in melanoma. Melanocytes and two melanoma lines, WM164 and WM35, expressed normal level E-cadherin and Desmoglein 1, whereas most melanomas (18 out of 20) expressed no E-cadherin and significantly reduced Desmoglein 1. Overexpression of dominant negative E-cadherin and Desmoglein 1 in melanocytes demonstrated that both molecules contribute to adhesion between melanocytes and keratinocytes. In contrast to melanocytes, most melanomas expressed HGF. All melanocytic cells expressed the HGF receptor c-Met, and autocrine HGF caused constitutive activation of c-Met, MAPK and P13 K. When autocrine activation was induced with HGF-expressing adenovirus, E-cadherin and Desmoglein 1 were decreased in melanocytes, WM164 and WM35. MAPK inhibitor PD98059 and P13K inhibitor wortmannin partially blocked the downregulation, suggesting that both pathways are involved in this process. c-Met was coimmunoprecipitated with E-cadherin, Desmoglein 1 and Plakoglobin, suggesting that they form a complex(es) that acts to regulate intercellular adhesion. Together, the results indicate that autocrine HGF decouples melanomas from keratinocytes by downregulating E-cadherin and Desmoglein 1, therefore frees melanoma cells from the control by keratinocytes and allows dissemination of the tumor mass.

### Example 10

### Induction of E-Cadherin Expression in Melanoma Cells Restores Regulatory Dominance of Keratinocytes over the Malignant Cells

Expression of E-cadherin by melanocytes is necessary in order for growth, proliferation, and invasiveness of the melanocytes to be maintained under the control of keratinocytes. Normal human melanocytes, keratinocytes, fibroblasts, and primary and metastatic melanoma cells were isolated and cultured as described (Hsu et al., 1996, In: Human Cell Culture Protocols, Humana Press Inc., Totowa, N.J., pp. 9-20; Boyce et at., 1985, J. Tissue Cult. Meth. 9:83-93). Melanocytes were mixed with keratinocytes at ratios of 1:5 to 1:10, and the mixtures were then seeded into 8-well chambers slides (LAB-TEK™, Nuns, Inc., Naperville, Ill.). The mixtures were maintained for four days, and then the cells were fixed with 3% (ELB) paraformaldehyde and permeabilized with a solution of 0.5% NP-40 detergent (nonidet P-40; octylphenoxypolyethoxyethanol; Sigma Chemical Co., St. Louis, Mo.). Fixed and permeabilized cells were subjected to double immunofluoresence analysis. In this analysis, the cells were contacted with a suspension of an antibody designated Mel-5 (Signet, Dedham, N4A) which binds specifically with TRP-1an melanoma cells, and were then contacted with a suspension. of Cy3™-conjugated (i.e. fluorescently-labeled) goat anti-mouse IgG (obtained from Jackson Immuno Research Laboratories, Inc., West Grove, Pa.). Next, the fixed and permeabilized cells were contacted with a biotinylated monoclonal antibody designated SAP (Hsu et al., 1998, Am. J. Pathol. 153:1435-1442) which binds specifically with the β3 subunit of the vitronectin receptor, and then with an antibody which binds specifically with Mel-CAM (Shih et al., 1994, Cancer Res. 54:2514-2520). The cells were then contacted with streptavidin conjugated with fluorescein isothiocyanate (FITC; Jackson ImmunoResearch Laboratories Inc., West Grove-, Pa.). Following these treatments, all melanoma cells were labeled with Cy3, and melanoma cells which expressed either or both of Mel-CAM and the β3 subunit of the vitronectin receptor were also labeled with FITC. For cell growth experiments, cell culture slides were counter-stained using Hoechst reagent (bisbenzimide; a cell nucleus stain; Sigma Chemical Co., St. Louis, Mo.). As a negative control, normal mouse serum was used in place of primary (i.e. anti-Mel-CAM and anti-β3 antibodies. Cell growth on the slides was monitored by counting cells in high power (250×) microscopic fields.

Melanoma cells (cell lines WM115 and three others i.e. cell lines which did not express E-cadherin) were transduced using an adenovirus vector which comprised a polynucleotide encoding E-cadherin (vector E-cad/Ad5) or a polynucleotide encoding LacZ protein (vector LacZ/Ad5). Vector E-cad/Ad5 was generated by transducing E1-positive 293 cells with a recombinant shuttle vector comprising full-length human E-cadherin cDNA and the vector pAd.CMV-Link.1 wherein the region E3 was deleted in the dl7001 human adenoviral DNA. The vector LacZ/Ad5 was constructed similarly, using a DNA encoding LacZ in place of the E-cadherin cDNA. Melanoma cells were transduced by maintaining the cells with 20 plaque forming units per cell of the selected vector for two hours in serum-free Dulbecco's modified Eagle's medium (DMEM). After 48 hours, the vector-treated cells were incubated for four hours with a 10 mg/ml solution of the fluorescent dye DiI (Molecular Probes, Eugene, Oreg.). The cells were detached from their growth substrate by contacting the substrate for 30 minutes with a 0.01% (w/v) suspension of trypsin in 1 mM Ca-HEPES-buffered salt solution (BBSS) at 37° C. Under these conditions, cadherins are specifically protected from proteolytic digestion. E-cadherin expression was detected using flow analysis/cell sorting procedure in which cells were contacted with a fluorescently-labeled anti-E-cadherin antibody.

For adhesion-blocking experiments, melanoma cells infected with E-cad/Ad5 were contacted at 4° C. for 30 minutes with a 5 μg/ml suspension of a monoclonal antibody designated SHE78-7 (Sigma Chemical Co., St. Louis, Mo.), which binds specifically with E-cadherin. The cells were rinsed with HBSS and resuspended in an assay medium comprising 1% (w/v) bovine serum albumin and 1 mM calcium in HBSS. A total of about 2×10⁵ cells in a volume is of 400 μl was added to differentiated keratinocyte monolayers in 4-well chamber slides. Keratinocyte differentiation in the monolayers had been induced prior to contact with the melanoma cell suspension by treatment of the cells with 2 mM calcium for 1 hour, as described by Valyi-Nagy et al. (1993, Lab. Invest. 69:152-159). The melanoma cell suspension was maintained in contact with the monolayers for 1 hour, in order to allow adherence of melanoma cells to keratinocytes. After this period, non-adherent cells were removed, and the slices were fixed using 3% (v/v) paraformaldehyde. The numbers of adherent cells per high power (250×) microscopic field were assessed in triplicate wells using a fluorescence microscope. Statistical analyses were performed using the Student's t-test.

Invasiveness of melanoma cells was assessed in artificial skin reconstructs, as described by Hsu et al. (1998, Am. J. Pathol., 153:1435-1442). Human foreskin dermal fibroblasts suspended in rat tail collagen were placed onto a pre-cast collagen gel and allowed to constrict the collagen for six days. E-cad/Ad5-transduced, LacZ/Ad5-transduced, and non-transduced melanoma cells (cell line 1205Lu) were then mixed with epidermal keratinocytes at a 1:5 ratio and seeded onto the surface of the dermal constructs. Five days later, cultures were lifted to the air-liquid interface in order to allow stratification of epidermal keratinocytes. Ten days thereafter, the reconstructs were harvested, fixed using paraformaldehyde, embedded in paraffin, sectioned, and stained with hematoxylin and eosin. Apoptosis in the reconstructs was assessed using the ApopTag™ in situ apoptosis detection kit (Oncor, Gaithersburg, Md.) according to the manufacturer's instructions.

It is known that melanocytes, but not melanoma cells, express the adhesion receptor designated E-cadherin (Hsu et al., 1996, J. Invest. Dermatol. Symp. Proc. 1:188-194). However, the role of E-cadherin in the failure of keratinocytes to regulate melanoma cells has not previously been appreciated.

In the experiments described herein, melanoma cell lines which do not express E-cadherin were transduced with an adenovirus construct which comprised an E-cadherin CDNA (E-cad/Ad5) or a LacZ-encoding DNA (LacZ/Ad5). Cells transduced with E-cad/Ad5 expressed E-cadherin, and neither cell transduced with LacZ/Ad5 nor non-transduced cells expressed E-cadherin. Expression of E-cadherin in cells transduced with E-cad/Ad5 was confirmed by Western blotting using a fluorescent antibody which binds specifically with E-cadherin. The functionality of E-cadherin expressed in these cells was confirmed by observing a 4-fold increase in adhesion of transduced melanoma cells to keratinocytes, relative to cells transduced with LacZ/Ad5 and relative to non-transduced cells. In addition, adherence of E-cad/Ad5-transduced cells was significantly reduced in the presence of an antibody which binds specifically with E-cadherin.

Normal (i.e. non-melanoma) melanocytes mixed with normal human keratinocytes at a 1:5 or 1:10 ratio maintained the corresponding ratio over a seven day period of observation, despite proliferation of cells of both types. Melanoma cells transduced with E-cad/Ad5 also maintained a substantially constant ratio with co-cultured keratinocytes. Melanoma cells transduced with LacZ/Ad5, however, did not maintain a constant melanoma cell:keratinocyte ratio.

In order to determine whether E-cadherin contributes to the phenotypic plasticity of melanocytes in response to control exerted by contact of those cells with keratinocytes cell surface antigen expression was tested in E-cad/Ad5-transduced cells which were cultured in the presence of keratinocytes. Non-transduced and LacZ/AD5-transduced melanoma cells exhibited no detectable change in expression of melanoma-associated antigens such as the cell to cell adhesion molecule designated Mel-CAM/MUC18 or the β3 subunit of the ανβ3 vitronectin receptor. However, expression of these two antigens could not be detected in melanoma cells transduced with E-cad/Ad5 after seven days of co-culture with keratinocytes. E-cad/Ad5 transduced melanoma cells which were cultured in the absence of keratinocytes exhibited no change in Mel-CAM or β3 subunit antigen expression, relative to non-transduced cells. These results demonstrate that the presence of E-Cadherin on the surface of melanocytes is necessary for exertion of contact mediated control of keratinocytes over the phenotype of melanocytes.

The physiological significance of down-regulation of tumor-associated cell-surface antigens Mel-CAM and β3 on melanoma cell phenotype was demonstrated using a three-dimensional reconstruct model of human skin. Each reconstruct comprised a dermal compartment of fibroblasts which was separated by a basement membrane from an epidermal compartment comprising melanocytes and keratinocytes. Using this skin reconstruct model, it was determined that non-transduced melanoma cells and melanoma cells transduced with LacZ/Ad5 grew deep into the dermal compartment, forming strands of cell nests. In contrast, melanoma cells transduced with E-cad/Ad5 remained in the epidermal compartment and the upper portion of the dermal compartment. Furthermore, those E-cad/Ad5-transduced cells which were located in the upper portion of the dermal compartment exhibited typical signs of apoptotic cell death, including nuclear condensation and apoptotic body formation. Free 3′-hydroxy ends resulting from DNA fragmentation were detected in these cells using a commercially available apoptosis detection kit. Invasive melanoma cells in control reconstructs did not exhibit signs of apoptosis.

### Example 11

### Gene Regulation Due to Cadherin Expression

Enhanced expression of cadherin in melanoma cells resulting in restoration of keratinocyte control over melanoma cell proliferation, growth and dendricity. Specific genes have also been identified which are upregulated and down regulated with cadherin expression.

TABLE 1
Genes which are up regulated with cadherin expression.

Homo sapiens growth arrest and DNA-damage-inducible protein
GADD45beta mRNA
Human mRNA for chemokine
Mucin 1
Human MEK5 mRNA
FK506-binding protein 12kDa (hFKBP-12)
inositol 1,4,5-triphosphate 3-kinase
p cadherin
Human DNase 1 homolog (DNAS1L2) mRNA
Homo sapiens mRNA for KIAA0754 protein
Human BLu protein (BLu) mRNA
Human anti-mullerian hormone type II receptor precursor
gene
Homo sapiens mRNA; cDNA DKFZp586J1923
Homo sapiens X-arrestin mRNA
Human normal keratinocyte mRNA
Homo sapiens, alpha-2 (VI) collagen
Homo sapiens brain expressed ring finger protein mRNA
Human presenilin I-374 (AD3-212) mRNA
Human mRNA for retinal S-antigen
Homo sapiens cDNA, 3 end /clone=IMAGE-356646
Homo sapiens mRNA; cDNA DKFZp586G1923
Homo sapiens apoptosis associated protein (GADD34) mRNA
Novel human mRNA similar to mouse tuftelin-interacting
protein 10 mRNA
Homo sapiens ras interactor (RIN1) mRNA
V-Erba Related Ear-3 Protein
blk=protein tyrosine kinase
H.sapiens E48 gene
Human N-formylpeptide receptor (fMLP-R98) mRNA
Homo sapiens DNA from chromosome 19-cosmid R30879
containing USF2
TLS/CHOP=hybrid gene {translocation breakpoint}
Homo sapiens cDNA /gb=W28230
Human mRNA for tyrosine kinase
Human growth arrest and DNA-damage-inducible protein
(gadd45) mRNA
ets-2 mRNA
Homo sapiens mRNA for protein kinase Dyrk1B
Homo sapiens serine threonine kinase 11 (STK11) mRNA
Homo sapiens mRNA for Sck
H.sapiens MN1 mRNA
Human pancreatic mucin mRNA
beta nerve growth factor
Protein Kinase Ht31, Camp-Dependent
Homo sapiens mRNA for Natural killer cell p44-related gene
1 (NKp44RG1)
Homo sapiens mRNA for HUMAN P2XM
Homo sapiens clone 24778 unknown mRNA
Human hemopoietic cell protein-tyrosine kinase (HCK) gene
Human Bullous pemphigoid autoantigen BP180 gene
H.sapiens seb4B mRNA
Human mRNA for BST-2
Homo sapiens cDNA, 3 end /clone=IMAGE-2263415
Oncogene Tls/Chop, Fusion Activated
Human tumor necrosis factor-inducible (TSC-14) mRNA
Human beta-2-adrenergic receptor mRNA
Homo sapiens zinc finger homeodomain protein (ATBF1-A) mRNA
Homo sapiens mRNA for trithorax homologue 2
H.sapiens zinc finger transcriptional regulator mRNA
Human Chromosome 16 BAC clone CIT9S7SK-A-589H1
Homo sapiens regulator of G protein signaling 12 (RGS12)
gene
Homo sapiens novel antagonist of FGF signaling (sprouty-1)
mRNA
Homo sapiens TTF-I interacting peptide 21 mRNA
Homo sapiens cDNA, 3 end /clone=IMAGE-2500528
beta nerve growth factor
Human SH2-containing inositol 5-phosphatase (hSHIP) mRNA
Human lecithin-cholesterol acyltransferase mRNA
Homo sapiens mRNA; cDNA DKFZp434M0918
H.sapiens mRNA for leukocyte adhesion glycoprotein p150,95
Human PSF-2 mRNA, complete cds /cds=(96,2207)
Homo sapiens multidrug resistance-associated protein 3B
(MRP3) mRNA
Human guanylate binding protein isoform I (GBP-2) mRNA
H.sapiens SCA1 mRNA for ataxin
neurogenin 3
Homo sapiens lysosphingolipid receptor Edg5 mRNA
Homo sapiens mRNA for hSOX20 protein
Homo sapiens mRNA for KIAA0860 protein
Endothelial Cell Growth Factor 1
Homo sapiens KIAA0404 mRNA
Homo sapiens retinol dehydrogenase gene
Human lysosomal-associated multitransmembrane protein
(LAPTm5) mRNA
Homo sapiens cDNA /gb=W29115
Homo sapiens dynein light intermediate chain 2 (LIC2) mRNA
Human U2AF1-RS2 mRNA
Human secretogranin II gene
Human mRNA for KIAA0343 gene
Human 47-kD autosomal chronic granulomatous disease protein
mRNA
Homo sapiens cDNA, 3 end /clone=IMAGE-2394055
Homo sapiens cDNA
Human DEAD-box protein p72 (P72) mRNA
Human 130-kD pemphigus vulgaris antigen mRNA
Human G-alpha 16 protein mRNA
H.sapiens Staf50 mRNA
Human CX3C chemokine
Homo sapiens CLDN14 gene
Homo sapiens mRNA for KIAA0809 protein
glycine cleavage system T-protein
Homo sapiens laminin-related protein (LamA3)
Guanine Nucleotide Exchange Factor 2
Homo sapiens cDNA /gb=W28729
Homo sapiens putative ATP-dependent mitochondrial RNA
helicase (SUV3) mRNA
erythroblastosis virus oncogene homolog 2 protein (ets-2)
gene
Human bile salt-activated lipase (BAL) mRNA
Human DNA sequence from clone 221G9 on chromosome 22q11.2-
12.2
Homo sapiens orexin receptor-1 mRNA
Homo sapiens beta-casein (CSN2) gene
Human polymorphic epithelial mucin core protein mRNA
axin (AXIN)
Homo sapiens cDNA /gb=U51712
Human BENE mRNA
Human B61 mRNA
Homo sapiens mRNA for KET protein
VAV2=VAV oncogene homolog
Human AML1 mRNA for AML1b protein
interferon regulatory factor 1 gene
untitled /cds=(98,6199)
Homo sapiens mRNA; cDNA DKFZp586L012
Id1
Homo sapiens NF-AT3 mRNA
Human heregulin-beta2 gene
Human putative transmembrane receptor IL-1Rrp mRNA
laminin
Claudin-7
sarcolectin
Tubulin, Alpha 1, Isoform 44
Homo sapiens mRNA; cDNA DKFZp566J123
Homo sapiens mRNA for KIAA0710 protein
Homo sapiens growth-arrest-specific protein (gas) mRNA
H.sapiens mRNA for carnitine palmitoyltransferase I type II
Human stratum corneum chymotryptic enzyme mRNA
Homo sapiens mRNA for serine protease (TLSP)
H.sapiens hR-PTPu gene for protein tyrosine phosphatase
brain-expressed HHCPA78 homolog
Homo sapiens keratin 16
Human mRNA for integrin beta 4
H.sapiens TROP-2 gene
plakoglobin (PLAK) mRNA
Human Wiskott-Aldrich syndrome protein (WASP) mRNA
Homo sapiens basic-helix-loop-helix-PAS orphan MOP3 (MOP3)
mRNA
Human interferon-inducible protein 9-27 mRNA
Homo sapiens mRNA for KIAA0284 gene
Human mRNA for KIAA0346 gene
Homo sapiens mRNA for hTCF-4
H.sapiens mRNA for leucine zipper protein
Human polymorphic epithelial mucin (PEM)
Homo sapiens cDNA, 3 end /clone=IMAGE-1420488
Homo sapiens CDNA, 5 end /clone=IMAGE-359747
Human squamous cell carcinama of esophagus mRNA for GRB-7
SH2 domain protein
Novel human gene mapping to chomosome 22 /cds=(372,1532)
Human placental tissue factor (two forms)
uPA gene
Human flt3 ligand
Human amphiregulin (AR) mRNA
Homo sapiens connexin 31 (GJB3) gene
Cluster Incl. Y00503:Human mRNA for keratin 19
/cds=(32,1234) /gb=Y00503 /gi=34038 /ug=Hs.182265 /len=1360
H.sapiens keratinocyte transglutaminase gene
Human mRNA for KIAK0002 gene
H.sapiens mRNA for interleukin-1 receptor antagonist
Homo sapiens normal epithelial cell-specific 1 (NES1) gene
Homo sapiens mRNA for Musashi
Homo sapiens Polycomb 2 homolog (hPc2)
JM3 preprotein translocase
keratin 13
E-cadherin, exon 3 and joined CDS
Homo sapiens VEGF165
IFN-beta 2a
alpha-tubulin
Desmoplakin I (DPI)
Human bullous pemphigoid antigen (BPAG1)
Homo sapiens megsin mRNA
Human activating transcription factor 3 (ATF3)
Human mRNA for vascular anticoagulant-beta (VAC-beta)
Homo sapiens stanniocalcin-related protein mRNA
Homo sapiens cDNA, 3 end /clone=IMAGE-1571997
Homo sapiens cDNA /clone=IMAGE-916052
Human mRNA for cytokeratin 15
Homo sapiens Dickkopf-1 (hdkk-1)
H.sapiens mRNA (clone 9112)
Human mRNA for antileukoprotease (ALP) from cervix uterus
Homo sapiens ataxia-telangiectasia group D-associated
protein mRNA
Homo sapiens cDNA, 3 end /clone=IMAGE-301715
Homo sapiens hCPE-R mRNA for CRE-receptor
Homo sapiens 195 kDa cornified envelope precursor mRNA
H.sapiens gene for cytokeratin 17
Homo sapiens CD24 signal transducer mRNA
Human mRNA for Arg-Serpin (plasminogen activator-inhibitor
2, PAI-2)
H.sapiens CL 100 mRNA for protein tyrosine phosphatase
Homo sapiens cDNA, 3 end /clone=IMAGE-1089034
Human keratin type 16 gene
Human maspin mRNA
Homo sapiens cDNA, 5 end /clone=IMAGE-587049
Huma elafin gene
Homo sapiens cDNA, 3 end /clone_end=3
Human maspin mRNA
Homo sapiens cDNA, 3 end /clone=IMAGE-2090244
Human thymosin beta-4 mRNA
H.sapiens mRNA (clone 9112) /cds=(165,911)
Human mRNA for lipocortin
H.sapiens CaN19 mRNA
Human small proline rich protein (sprI) mRNA
50 kDa type I epidermal keratin gene
Homo sapiens cDNA, 3 end /clone=IMAGE-2443791
Human gastrointestinal tumor-associated antigen GA733-1
protein gene
Homo sapiens cDNA, 3 end /clone=IMAGE-345592
Human keratin type II (58 kD)
Homo sapiens cDNA, 3 end /clone=IMAGE-1735496
Homo sapiens keratin 6 isoform KGe (KRT6E)
Human uvomorulin (E-cadherin) (UVO)
nucleotide diphosphate kinase B
Tissue inhibitor of metalloproteinases 3 (TIMP3)
interferon-inducible protein 9-27
14-3-3 sigma
MEKK3
JNK2
c-myc
integrin beta 4
SOD1

TABLE 2
Genes which are down regulated with cadherin expression.

tegrin alpha-4 subunit Human melanocyte-specific gene 1
(msg1) mRNA
Human putative potassium channel subunit (h-erg) mRNA
Human insulin-like growth factor binding protein 6 (IGFBP6)
sodium/myo-inositol cotransporter (SLC5A3) gene
Human S100 protein beta-subunit gene, exon 3
Ras-like GTP-binding protein REM mRNA
Human mRNA for cytochrome P-450 (11 Beta)
Human G protein-activated inwardly rectifying K+ channel
(GIRK4)
Homo sapiens cadherin-4 mRNA
GTPase-activating protein
Human L-myc
Human neuronal PAS1 (NPAS1)
S100E calcium binding protein
frizzled-1
Human tumor necrosis factor and lymphotoxin genes
Homo sapiens mRNA for metalloproteinase
Homo sapiens GATA-4 mRNA
Homo sapiens telomerase reverse transcriptase (hTRT) mRNA
Homo sapiens HOX11L1 gene
Human cdc2-related protein kinase mRNA
Human G protein gamma-4 subunit mRNA
membrane-type matrix metalloproteinase
Human Bcl-2 related (Bfl-1) mRNA
Human Wnt10B
Homo sapiens TWIK-related acid-sensitive K+ channel (TASK)
mRNA
Human phospholipase c delta 1 mRNA
Human mRNA for transcription factor AREB6
adrenomedullin precursor
Homo sapiens mRNA; cDNA DKFZp566D1146
Human DNA sequence from clone 1183I21 on chromosome 20q12.
rhoC
uridine phosphorylase
lactate dehydrogenase M chain
A06g MMP10
IL1
DNA recomb and repair protein HNGS1
guanylate kinase (GMP kinase)
early growth response protein 1
DNA repair ERCC1
transforming protein rhoA H12
alpha-catenin related protein

What is claimed is:

1. A method of preventing proliferation of melanoma cells comprising contacting melanoma cells with an agent which modulates expression of E-cadherin in melanoma cells so that keratinocyte control over melanoma cell proliferation is restored.

2. A method of preventing proliferation of melanoma cells comprising contacting melanoma cells with an agent which modulates the HGF autocrine loop in melanoma cells so that down regulation of E-cadherin and Desmoglein 1 and uncontrolled proliferation of said melanoma cells are prevented.

3. A method of treating melanoma comprising administering an effective amount of an agent to melanoma cells which prevents constitutive activation of c-Met, MAPK, AKT or PI3K in said melanoma cells.

4. The method of claim 2 wherein the agent is an HGF blocking antibody.

5. The method of claim 2 wherein the agent is an antisense oligonucleotide.

6. A method of treating melanoma comprising administering an effective amount of an agent to melanocytes or melanoma cells which prevents prolonged HGF stimulation and down regulation of E-cadherin and Desmoglein 1.

7. A method of treating melanoma comprising administering an effective amount of an agent to melanocytes or melanoma cells which down regulates HGF in melanoma cells.

8. A method for identifying an agent which modulates expression of E-cadherin in melanoma cells comprising contacting melanoma cells with a test agent and determining whether keratinocytes control over melanoma cell proliferation is restored.

9. A method of preventing proliferation of melanoma cells comprising contacting melanoma cells with an agent which modulates expression of cadherin in melanoma cells wherein genes which control or transmit physiologically relevant signals from keratinocytes to melanocytes are upregulated or down regulated so that control over melanoma cell proliferation is restored.
